(19) United States
(12) Patent Application Publication
(10) Pub. No.: US 2022/0040356 A1
(43) Pub. Date: Feb. 10, 2022

(54) SPACE CLEANING SYSTEM AND SPACE CLEANING METHOD

(71) Applicant: PANASONIC INTELLECTUAL PROPERTY MANAGEMENT CO., LTD., Osaka (JP)

(72) Inventors: Tsutomu ICHIHARA, Osaka (JP); Noriaki FUKUMOTO, Osaka (JP)

(21) Appl. No.: 17/276,733

(22) PCT Filed: Aug. 27, 2019
(86) PCT No.: PCT/JP2019/033499

§ 371 Date: Mar. 16, 2021

(30) Foreign Application Priority Data
Sep. 21, 2018 (JP) 2018-177714
Oct. 25, 2018 (JP) 2018-201195

(51) Int. Cl. A61L 9/14; F24F 7/007; F24F 11/70

(52) U.S. Cl. CPC A61L 9/14; F24F 7/007; A61L 2209/13; A61L 2209/11; F24F 11/70

(57) ABSTRACT

A space cleaning system includes: a detection unit configured to detect generation and a generation position of an infectious substance in a space; an airflow control unit configured to generate an airflow towards the generation position detected in response to detection of the generation of the infectious substance; and a cleaning control unit configured to clean the space at least after the generation of the infectious substance is detected.

### TECHNICAL FIELD

The present invention relates to a space cleaning system and a space cleaning method for suppressing the spread of infection by an infectious substance.

### BACKGROUND ART

Technologies for suppressing the spread of infection by an infectious substance have been suggested. Patent Literature (PTL) 1 discloses a technology that can narrow down those who are suspected of being infected.

### CITATION LIST

### Patent Literature

PTL 1: Japanese Unexamined Patent Application Publication No. 2017-117416

PTL 2: Japanese Unexamined Patent Application Publication No. 2013-52784

### SUMMARY OF THE INVENTION

### Technical Problem

The present invention provides a space cleaning system and a space cleaning method capable of suppressing formation of a region at a high risk of infection by an infectious substance in space.

### Solutions to Problem

A space cleaning system according to one aspect of the present invention includes: a detection unit configured to detect generation and a generation position of an infectious substance in a space; an airflow control unit configured to generate an airflow towards the generation position detected in response to detection of the generation of the infectious substance; and a cleaning control unit configured to clean the space at least after the generation of the infectious substance is detected.

A space cleaning method according to another aspect of the present invention includes: detecting generation and a generation position of an infectious substance in a space; generating an airflow towards the generation position detected in response to detection of the generation of the infectious substance; and cleaning the space at least after the generation of the infectious substance is detected.

### Advantageous Effect of Invention

Realized with the present invention are a space cleaning system and a space cleaning method capable of suppressing formation of a region at a risk of infection by an infectious substance in space.

### BRIEF DESCRIPTION OF DRAWINGS

FIG. 1 is a block diagram illustrating a functional configuration of a space cleaning system according to an embodiment.

FIG. 2 is a diagram illustrating one example of a space to which the space cleaning system according to the embodiment is applied.

FIG. 3 is a flowchart of Operation Example 1 of the space cleaning system according to the embodiment.

FIG. 4 is a diagram illustrating a space where high concentration of an infectious substance are generated.

FIG. 5 is a diagram illustrating a space where the concentration of the infectious substance is diluted.

FIG. 6 is a diagram illustrating a change in the concentration of the infectious substance when an airflow is generated toward the generation position of the infectious substance.

FIG. 7 is a flowchart of Operation Example 2 of the space cleaning system according to the embodiment.

FIG. 8 is a flowchart of Operation Example 3 of the space cleaning system according to the embodiment.

### DESCRIPTION OF EXEMPLARY EMBODIMENTS

Hereinafter, the embodiments will be described in detail with reference to the drawings. Note that the embodiments described below illustrates a comprehensive or detailed example. Numerical values, shapes, materials, components, arrangement positions and connection modes of the components, steps, a sequence of the steps, etc. are each one example, and are not intended to limit the present invention. Moreover, of the components in the embodiments below, those not described in an independent claim will be described as optical components.

Note that each of the drawings is a schematic diagram and does not necessarily provide a precise illustration. In each of the drawings, those with the substantially same configurations will be marked with same signs and a description thereof may be omitted or simplified in some cases.

### Embodiment

### [Configuration of Space Cleaning System]

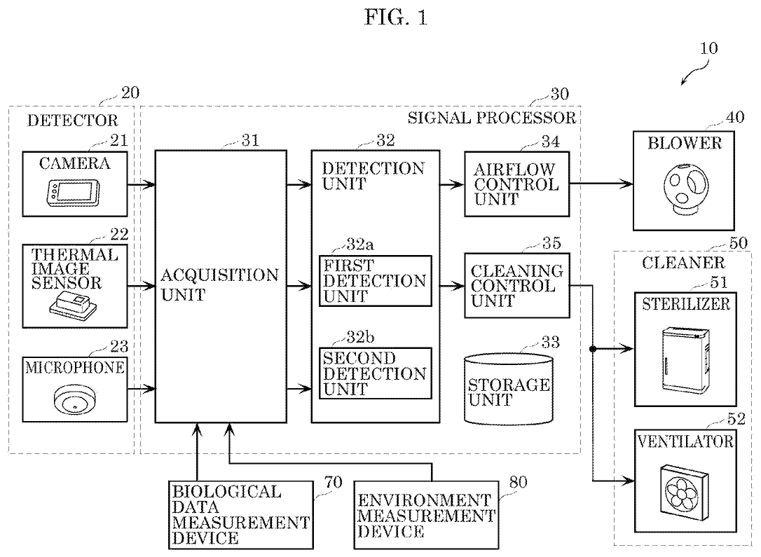

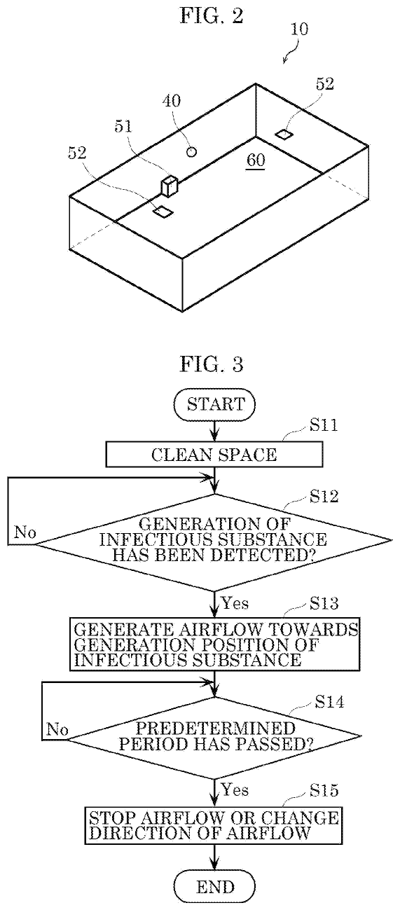

First, the configuration of the space cleaning system according to the embodiment will be described. FIG. 1 is a block diagram illustrating a functional configuration of the space cleaning system according to the embodiment. FIG. 2 is a diagram illustrating one example of a space to which the space cleaning system according to the embodiment is applied.

Space cleaning system 10 according to the embodiment is a system which performs processing (hereinafter also written as injection suppression processing) for suppressing the infection of people staying in space 60 with an infectious substance. Space 60 is, for example, a closed space of, for example, nursing facilities, hospitals, hospital waiting rooms, etc. and a room without any partition or the like. The infectious substance is, for example, mold, bacteria, and viruses. As illustrated in FIG. 1, space cleaning system 10 includes detector 20, signal processor 30, blower 40 and cleaner 50, and biological data measurement device 70 and environmental measurement device 80.

Detector 20 is a device for detecting the generation position of the infectious substance in space 60. Here, detector 20 includes: camera 21 which photographs an image (more specifically, a moving image or a still image) in space 60; thermal image sensor 22 which photographs a thermal image in space 60; microphone 23 which acquires voice in space 60; etc. Detector 20 may plurally include camera 21, thermal image sensor 22, and microphone 23. Note that space cleaning system 10 is only required to include at least one detector 20.

Signal processor 30 is a device which detects the generation and generation position of the infectious substance in space 60 based on at least one of image data, thermal image data, and voice data obtained from detector 20, and controls blower 40 and cleaner 50 based on results of the detection. Signal processor 30 is realized by, for example, a microcomputer or a processor. A detailed configuration of signal processor 30 will be described later on.

Blower 40 is a device which generates an airflow towards the generation position of the infectious substance. Blower 40 operates based on a control signal outputted from signal processor 30 (for example, airflow control unit 34). Blower 40 is a blower, for example, a circulator having relatively high directionality but may be an air conditioner.

Note that space cleaning system 10 may include a plurality of blowers 40. Space cleaning system 10 may include at least one blower 40. In an example of FIG. 2, blower 40 is installed along the wall defining space 60.

Cleaner 50 is a device which performs cleaning of space 60. Cleaner 50 operates based on a control signal outputted from signal processor 30 (for example, cleaning control unit 35). Cleaning here means cleaning in a broad sense and includes not only disinfecting or sterilizing the infectious substance with hypochlorous acid or the like but also, for example, discharging the infectious substance to an outside of space 60 through ventilation.

Cleaner 50 includes, for example, sterilizer 51 and ventilator 52. Sterilizer 51 is, for example, a device which discharges hypochlorite water but may be a device which collects the air, sterilizes the collected air by the hypochlorous acid, and discharges the air or any other sterilizer. In the example of FIG. 2, cleaner 50 is installed along the wall defining space 60.

Ventilator 52 is a device which performs at least one of exhaust and air supply. The exhaust means the discharge of the air in space 60 to an outside of space 60, and the air supply means intake of the air outside of space 60 into space 60. In the example of FIG. 2, ventilator 52 for exhaust and ventilator 52 for intake are attached to the ceiling defining space 60. More specifically, ventilator 52 is realized by, for example, a motor and a fan driven by the motor.

Note that cleaner 50 may include a plurality of sterilizers 51 and a plurality of ventilators 52. Space cleaning system 10 may include at least one cleaner 50.

Moreover, blower 40 and cleaner 50 are discriminated for the convenience of explanation but are not clearly discriminated from each other. For example, space cleaning system 10 may generate airflow by ventilator 52 or may perform cleaning by blower 40. Moreover, blower 40 and cleaner 50 may be realized by an integrated device (single device) having respective functions of blower 40 and cleaner 50.

Moreover, the generation of the airflow and the cleaning of space 60 may be realized by combining together a plurality of devices. For example, the cleaning of space 60 through ventilation may be realized by combining together blower 40 and a window opening and closing device (not illustrated) which opens and closes a window provided on the wall defining space 60.

Biological data measurement device 70 is a device which measures biological data of a person staying in space 60. For example, biological data measurement device 70 is a sensor which measures, as the biological data, the body temperature, the pulse rate, the heart rate, the respiratory rate, or the arterial oxygen saturation (SpO₂) of the person staying in space 60. Biological data measurement device 70 is, for example, a wearable type sensor fitted to the person staying in space 60, and for example, transmits biological data including current position information of the person to signal processor 30. Biological data measurement device 70 may be realized as one function of a portable terminal such as a smartphone or a tablet terminal. Note that thermal image sensor 22 described above is capable of measuring the body temperature of the person staying in space 60 and thus may be included in biological data measurement device 70.

Environment measurement device 80 is a device which measures environment data in space 60. Environment measurement device 80 includes: for example, a temperature sensor which measures the temperature in space 60; a humidity sensor which measures the humidity in space 60; an air volume sensor which measures at least one of the air direction and the air speed in space 60; a CO₂ concentration sensor which measures the carbon dioxide (CO₂) concentration in space 60; a microbial sensor which measures the amount (concentration) of microorganisms such as molds or fungi; and a virus sensor which measures the amount (concentration) of viruses.

### [Configuration of Signal Processor]

Next, a detailed configuration of signal processor 30 will be described. Signal processor 30 includes acquisition unit 31, detection unit 32, storage unit 33, airflow control unit 34, and cleaning control unit 35.

Acquisition unit 31 is a communication circuit (in other words, communication interface) which acquires data from detector 20. More specifically, acquisition unit 31 acquires image data from camera 21, acquires thermal image data from thermal image sensor 22, and acquires voice data from microphone 23. Moreover, acquisition unit 31 acquires biological data from biological data measurement device 70 and acquires environment data from environment measurement device 80. Communication between acquisition unit 31, detector 20, biological data measurement device 70, and environment measurement device 80 may be wireless communication or wired communication. Communication standards are not specifically limited.

Detection unit 32 includes first detection unit 32a and second detection unit 32b. First detection unit 32a detects the generation and the generation position of the infectious substance in space 60 in real time based on data obtained from detector 20 via acquisition unit 31. That is, first detection unit 32a detects the generation timing and the generation position of the infectious substance in real time. Note that real time mentioned here does not provide precise meaning and may include a time lag of about several seconds.

For example, first detection unit 32a is capable of detecting, as the generation of the infectious substance, the operation of discharging the infectious substance (for example, hiccup or cough) by the person staying in space 60 through image recognition processing (for example, existing pattern matching processing) using the image data obtained from camera 21. Moreover, first detection unit 32a detects, as the generation position of the infectious substance, the position of a person who has performed the discharge operation. For example, an image recognition technology (for example, refer to PTL 2) of detecting, with a camera, a person who sneezes is known and such an image recognition technology is used for detecting the person who has performed the discharge operation.

Note that first detection unit 32a can improve the accuracy in detecting the generation position of the infectious substance by, for example, considering the orientation of a person who has performed the discharge operation, the posture of the aforementioned person, the position of the mouse of the aforementioned person, the distance over which discharged droplets typically reach, etc. Moreover, first detection unit 32a can also improve the accuracy in detecting the generation position of the infectious substance by considering the flow (air amount and air speed) of the air in space 60 when the discharge operation has been performed by use of the environment data measured by environment measurement device 80.

Moreover, first detection unit 32a can detect, as the generation of the infectious substance, the generation of voice from the operation of discharging the infectious substance by a person staying in space 60 through voice recognition processing using the voice data obtained from microphone 23. The voice from the discharge operation can be discriminated from other types of voice through, for example, machine learning. Moreover, a technology is known which estimates the arrival direction of voice (voice such as sneeze or cough in this case) by use of a plurality of microphones 23 such as a microarray. Through the aforementioned technology, first detection unit 32a can detect, as the generation position of infectious substance, the position of the person who has performed the discharge operation such as sneezing or coughing.

Moreover, first detection unit 32a may use the thermal image data obtained from thermal image sensor 22 to detect the person staying in the space. For example, first detection unit 32a specifies a person reflected on the thermal image based on the thermal image data through, for example, outline extraction processing (edge detection processing) to detect, as the generation of the infectious substance, that the specified people include a person whose body temperature is greater than or equal to a predetermined temperature (for example, 37.5 degrees Celsius). Moreover, first detection unit 32a detects the position of such a person as the generation position of the infectious substance.

Second detection unit 32b detects, from among the people staying in space 60, the suspicious person who is suspected to be infected with the infectious substance. For example, second detection unit 32b detects, as the suspicious person, the person whose temperature has been determined to be greater than or equal to a predetermined temperature based on the thermal image data. Second detection unit 32b can also detect the position of the suspicious person based on the thermal image data.

Moreover, second detection unit 32b may detect, as the suspicious person, the person who is estimated to have poor physical condition based on the biological data. Second detection unit 32b can also detect the position of the suspicious person based on current position information included in the biological data.

Moreover, second detection unit 32b may detect, as the suspicious person based on the number of times of discharge operation performed in a fixed period by the person staying in space 60, for example, the person whose number of times of the discharge operation in the fixed period is greater than or equal to a predetermined number of times.

According to results of the detection performed by second detection unit 32b, a configuration such that the discharge operation performed by a person who has good physical condition is not determined to be the generation of infectious substance. That is, it is possible to improve the accuracy in detecting the generation of infectious substance.

As described above, detection unit 32 may detect, as the generation position of the infectious substance in space 60 based on the results of the detection performed by first detection unit 32a, a place where the person has performed the discharge operation or may detect, as the generation position of the infectious substance in space 60 based on the results of detection performed by first detection unit 32a and the results of detection performed by second detection unit 32b, a place where the suspicious person has performed the discharge operation. In the latter case, comparison between the position of the person who has performed the discharge operation detected by first detection unit 32a and the position of the suspicious person detected by second detection unit 32b makes it possible to judge whether or not the discharge operation is performed by the suspicious person.

Note that whether or not the person staying in space 60 is the suspicious person may be detected based on information inputted to a portable terminal (not illustrated) possessed by the aforementioned person. That is, the person staying in space 60 may self-report whether or not he or she is the suspicious person. In this case, information indicating whether or not he or she is the suspicious person is transmitted together with the current position information from the portable terminal to signal processor 30.

Moreover, mutually different methods may be used for detecting the generation of the infectious substance and detecting the generation position of the infectious substance. For example, the voice data may be used for detecting the generation of the infectious substance and the image data may be used for detecting the generation position of the infectious substance.

Storage unit 33 is a storage unit which stores: for example, programs executed for detection unit 32, airflow control unit 34, and cleaning control unit 35 to perform signal processing; and information required for the signal processing. Storage unit 33 is realized by, for example, a semiconductor memory. Storage unit 33 also stores: for example, space information indicating the shape and size of space 60; and device arrangement information indicating the arrangement of blower 40 and cleaner 50 in space 60. The space information and the device arrangement information are inputted via, for example, a user interface device (not illustrated) included in space cleaning system 10.

Airflow control unit 34 generates an airflow towards the detected generation position of the infectious substance in response to the detection of the generation of the infectious substance. More specifically, airflow control unit 34 outputs a control signal to blower 40 to thereby control blower 40, causing blower 40 to blow the air towards the generation position of the infectious substance. More specifically, airflow control unit 34 can control the direction of the air blowing (in other words, the direction of the airflow) and the intensity of air blowing (in other words, the intensity of the airflow). The control signal may be outputted to blower 40 through wireless communication or may be outputted to blower 40 through wired communication.

Note that airflow control unit 34 may control the direction of the air blowing (in other words, the direction of the airflow) and the intensity of the air (in other words, the intensity of the airflow) in consideration of the flow (air amount and air speed) of the air in space 60 upon the generation of the airflow by use of the environment data measured by environment measurement device 80. Consequently, airflow control unit 34 can generate more accurate airflow.

Cleaning control unit 35 cleans space 60 at least after the detection of the generation of the infectious substance. More specifically, cleaning control unit 35 outputs a control signal to cleaner 50 to thereby clean space 60. The control signal may be outputted to cleaner 50 through wireless communication or may be outputted to cleaner 50 through wired communication.

### Operation Example 1

Next, Operation Example 1 of space cleaning system 10 will be described. FIG. 3 is a flowchart of Operation Example 1 of space cleaning system 10.

First, cleaning control unit 35 cleans space 60 (S11). For example, cleaning control unit 35 outputs the control signal to sterilizer 51 to thereby clean space 60 but may output the control signal to ventilator 52 to thereby clean space 60. Cleaning control unit 35 may clean space 60 by use of at least one of sterilizer 51 and ventilator 52.

Next, airflow control unit 34 determines whether or not the generation of an infectious substance has been detected by detection unit 32 (S12). A method for detecting the generation of an infectious substance by detection unit 32 is as described above but is not specifically limited. For example, a method that can detect the generation of relatively high concentration of an infectious substance (more specifically, a method for detecting operation of discharging an infectious substance) is used.

When it is determined that the generation of the infectious substance has not been detected by detection unit 32 (No in S12), only the cleaning of space 60 by cleaning control unit 35 is continued. On the other hand, when it is determined that the generation of the infectious substance has been detected by detection unit 32 (Yes in S12), airflow control unit 34 not only cleans space 60 by cleaning control unit 35 but also generates airflow towards the generation position of the infectious substance detected by detection unit 32 (S13). As described above, airflow control unit 34 immediately generates the airflow towards the generation position of the infectious substance, which is triggered by the detection of the generation of the infectious substance.

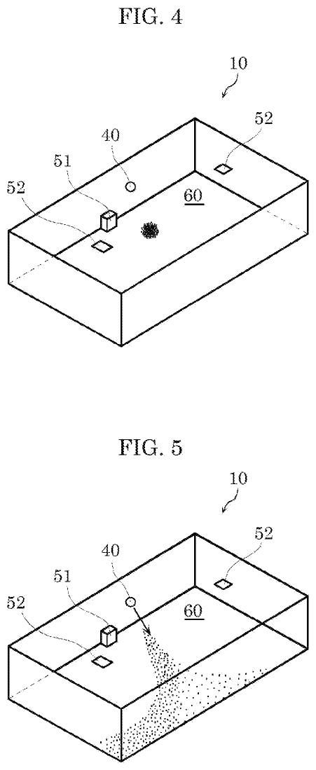

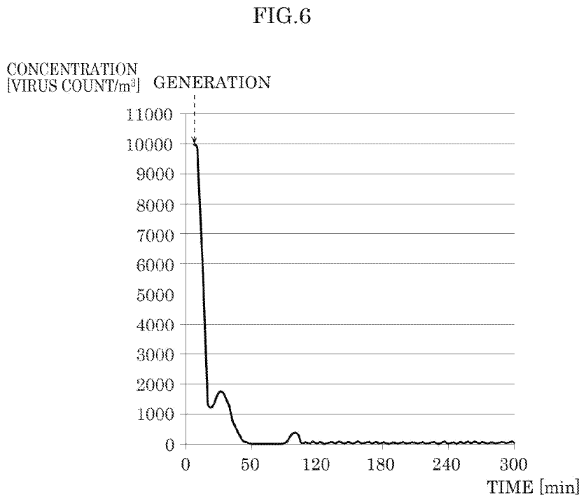

For example, performing the operation of discharging the infectious substance by a person generates high concentration of the infectious substance. FIG. 4 is a diagram illustrating space 60 where the high concentration of the infectious substance is generated. Black dots in space 60 in FIG. 4 indicate particles corresponding to the infectious substance. As a result of generating the airflow towards an estimated position where the high concentration of the infectious substance have been generated as described above, the concentration of the infectious substance is diluted. FIG. 5 is a diagram illustrating space 60 where the concentration of the infectious substance is diluted, with the black dots in space 60 illustrating the particles corresponding to the infectious substance. FIG. 6 is a diagram illustrating a change in the concentration of the infectious substance upon the generation of the airflow towards the generation position of the infectious substance.

As illustrated in FIGS. 5 and 6, the concentration of the infectious substance is diluted according to the generation of the airflow. FIG. 6 illustrates results of simulation of the concentration of the infectious substance around the generation position of the infectious substance when the air amount of blower 40 is 4.5 m³/min. As illustrated in FIG. 6, where the original concentration of infectious substance is 10000 viruses/m³, the concentration of infectious substance is 2000 viruses/m³ or less (that is, one fifth of the original concentration or less) 15 seconds after the start of air blowing. Note that a person who has suctioned particles containing 3600 viruses (that is, one example of the infectious substance) per hour is typically infected with the aforementioned viruses at a probability of 50%. Where the average human respiration amount is 6 litters/min, the probability that a person staying in space where the virus concentration is 10000 viruses for one hour is infected with the aforementioned virus is 50%.

As described above, if the concentration of the infectious substance is diluted by the airflow at the generation position of the infectious substance within a relatively short period, the formation of a region at a high risk of infection in space 60 is suppressed. Moreover, a relatively low overall concentration of the infectious substance in space 60 effectively cleans the infectious substance by cleaner 50, which can reduce the risk of infection.

The infectious substance is discharged by a person in many cases. That is, there is high possibility that a person is present at the generation position of the infectious substance. Therefore, continuously generating the airflow towards the generation position of the infectious substance possibly causes discomfort of the person.

Thus, airflow control unit 34 determines whether or not a predetermined period has passed since the airflow generation (S14), and stops the airflow or changes the direction of the airflow (S15) when it is determined that the predetermined period has passed (Yes in S14). More specifically, airflow control unit 34 outputs, to blower 40, a control signal instructing the stop of the air blowing or a control signal instructing a change in the direction of the air blowing. Note that when it is determined that the predetermined period has not passed (No in S14), airflow control unit 34 continues the generation of the airflow (air blowing) towards the generation position of the infectious substance. The predetermined period is, for example, 15 seconds, but is not specifically limited.

As described above, generating the airflow towards the generation position of the infectious substance in a limited period suppresses discomfort of the person with the airflow.

### Operation Example 2

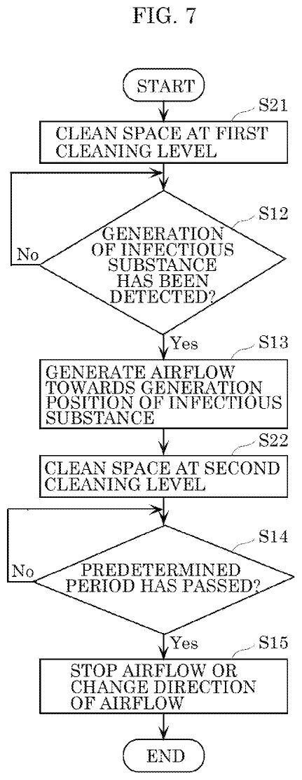

Cleaning control unit 35 may increase the cleaning level in response to the detection of the generation of the infectious substance to clean space 60. FIG. 7 is a flowchart of Operation Example 2 of space cleaning system 10 as described above. Note that Operation Example 2 will be described below, focusing on a difference from Operation Example 1 and those already described will be omitted as appropriate.

First, cleaning control unit 35 cleans space 60 at a first cleaning level (S21). Next, airflow control unit 34 determines whether or not the generation of the infectious substance has been detected by detection unit 32 (S12).

When it is determined that the generation of the infectious substance has not been detected by detection unit 32 (No in S12), the cleaning of space 60 at the first cleaning level is continued. On the other hand, when it is determined that the generation of the infectious substance has been detected by detection unit 32 (Yes in S12), airflow control unit 34 generates the airflow towards the generation position of the infectious substance (S13).

Moreover, when it is determined that the generation of the infectious substance has been detected by detection unit 32, cleaning control unit 35 cleans space 60 at a second cleaning level higher than the first cleaning level (S22). That is, cleaning control unit 35 increases the cleaning level in response to the detection of the generation of the infectious substance to clean space 60. The high cleaning level means that the concentration of the infectious substance can be reduced in shorter time.

For example, when the cleaning is performed by the discharge of hypochlorous acid by sterilizer 51, the cleaning level is adjusted depending on the discharge amount (concentration) of the hypochlorous acid. The discharge amount of the hypochlorous acid is greater in the cleaning performed at the second cleaning level than in the cleaning performed at the first cleaning level. Moreover, when the cleaning is performed by sterilizing the air collected by sterilizer 51 with the hypochlorous acid and discharging the aforementioned air and when the cleaning is performed through ventilation performed by ventilator 52, the cleaning level is adjusted depending on the air amount. The air amount is greater in the cleaning performed at the second cleaning level than in the cleaning performed at the first cleaning level. Note that the cleaning level is changed by the control signal outputted from cleaning control unit 35 to cleaner 50.

Increasing the cleaning level in response to the detection of the generation of the infectious substance as described above increases the power consumption of cleaner 50 only when the necessity of the cleaning is high, which therefore makes it possible to efficiently clean the infectious substance.

### Operation Example 3

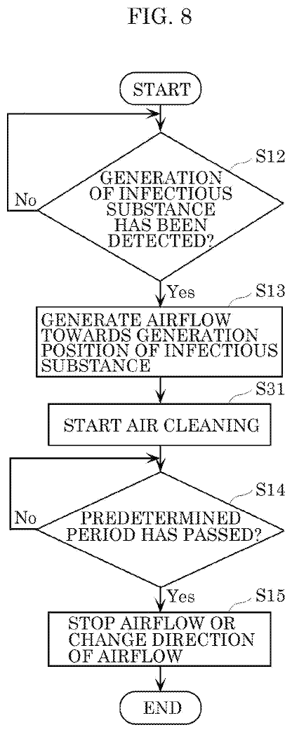

The cleaning of space 60 by cleaner 50 is performed on a regular basis in Operation Example 1. That is, cleaning control unit 35 continuously cleans space 60 from time before the generation of the infectious substance is detected to time after the generation of the infectious substance is detected. However, cleaning control unit 35 may start the cleaning of space 60 in response to the detection of the generation of the infectious substance. FIG. 8 is a flowchart of Operation Example 3 of such space cleaning system 10. Note that the description of Operation Example 3 will be given, focusing on a difference from Operation Example 1 and thus those already described will be omitted as appropriate.

In Operation Example 3, cleaner 50 is initially stopped. In this state, airflow control unit 34 determines whether or not the generation of the infectious substance has been detected by detection unit 32 (S12).

When it is determined that the generation of the infectious substance has been detected by detection unit 32 (Yes in S12), airflow control unit 34 generates the airflow towards the generation position of the infectious substance (S13).

Moreover, when it is determined that the generation of the infectious substance has been detected by detection unit 32, cleaning control unit 35 starts the cleaning of space 60 (S31).

Starting the cleaning in response to the detection of the generation of the infectious substance as described above increases the power consumption of cleaner 50 only when the necessity of cleaning is high, which therefore makes it possible to efficiently clean the infectious substance.

### Effects and Others

As described above, space cleaning system 10 includes: detection unit 32 which detects the generation and the generation position of an infectious substance in space 60; airflow control unit 34 which generates an airflow towards the detected generation position in response to the detection of the generation of the infectious substance; and cleaning control unit 35 which cleans space 60 at least after the detection of the generation of the infectious substance.

Such space cleaning system 10 can suppress the formation of a region at a high risk of infection with the infectious substance in space 60.

Moreover, airflow control unit 34 stops the generation of the airflow after the passage of a predetermined period since the generation of the airflow.

Such space cleaning system 10 can generate the airflow towards the generation position of the infectious substance in a limited period to thereby suppress discomfort felt with the airflow by the person.

Moreover, airflow control unit 34 changes the direction of the airflow after the airflow is generated towards the generation position.

Such space cleaning system 10 can generate the airflow towards the generation position of the infectious substance in a limited period to thereby suppress the discomfort felt with the airflow by the person.

Moreover, cleaning control unit 35 continuously cleans space 60 from time before the generation of the infectious substance is detected to time after the generation of the infectious substance is detected.

Such space cleaning system 10 can clean the infectious substance on a regular basis.

Moreover, cleaning control unit 35 increases the cleaning level in response to the detection of the generation of the infectious substance to clean space 60.

Such space cleaning system 10 can efficiently clean the infectious substance.

Moreover, cleaning control unit 35 starts the cleaning of space 60 after the detection of the generation of the infectious substance.

Such space cleaning system 10 can efficiently clean the infectious substance.

Moreover, cleaning control unit 35 controls sterilizer 51 to thereby clean space 60. Sterilizer 51 is one example of a device which sterilizes the air by use of hypochlorous acid.

Such space cleaning system 10 can clean space 60 by use of the hypochlorous acid.

Moreover, cleaning control unit 35 controls ventilator 52 to thereby clean space 60. Ventilator 52 is one example of a device which ventilates space 60.

Such space cleaning system 10 can clean space 60 through ventilation.

Moreover, an air cleaning method executed by a computer such as space cleaning system 10 includes: detecting the generation and the generation position of the infectious substance in space 60; generating an airflow towards the detected generation position in response to the detection of the generation of the infectious substance; and cleaning space 60 at least after the detection of the generation of the infectious substance.

Such a space cleaning method can suppress the formation of a region at a high risk of infection with the infectious substance in space 60.

### OTHER EMBODIMENTS

The embodiment has been described but the present invention is not limited to the embodiment described above.

For example, the space to be cleaned by the space cleaning system is not limited to the nursing facilities, hospitals, or hospital waiting rooms, etc. The space to be cleaned by the space cleaning system may be an airport. Moreover, the space to be cleaned by the space cleaning system is not limited to a building and may be a space in a mobile body such as a railroad or an aircraft.

Moreover, in the embodiment described above, the air amount for generating the airflow may be controlled. For example, with an increase in the distance from an installation position of the blower to the generation position of the infectious substance, the control of increasing the air amount may be more permitted.

Moreover, processing executed by a specific processing unit may be executed by another processing unit in the embodiment described above Moreover, the sequence of a plurality of types of processing may be changed and a plurality of types of processing may be executed in parallel.

Moreover, the components in the embodiment described above may be realized by executing software programs suitable for the respective components. Each of the components may be realized by reading out and executing a software program recorded on a recording medium such as a hard disc or a semiconductor memory by a program execution unit such as a CPU or a processor.

Moreover, each of the components may be realized by a hardware. Each component may be a circuit (or an integrated circuit). These circuits may be formed by one circuit as a whole or may be mutually separate circuits. Moreover, these circuits may be each a general-purpose circuit or a dedicated circuit.

Moreover, a comprehensive or detailed mode of the present invention may be realized by a system, a device, a method, an integrated circuit, a computer program, or a computer-readable recording medium such as a CD-ROM. Moreover, the mode of the present invention may also be realized by combining together the system, the device, the method, the integrated circuit, the computer program, and the recording medium in a desired manner.

For example, the present invention may be realized as a space cleaning method, may be realized as a program causing a computer to execute the space cleaning method, or may be realized as a computer-readable, non-transitory recording medium having such a program recorded thereon.

Moreover, in the embodiment described above, the space cleaning system is realized by a plurality of device but may also be realized as a single device. When the space cleaning system is realized by the plurality of devices, the components included in the space cleaning system described in the embodiment above may be allocated to the plurality of devices in any manner.

Moreover, the space cleaning system may be realized as a client server system. For example, the signal processor is realized as a server device and the detector, the blower, or the cleaner may also be realized as client devices.

In addition, the present invention includes: a mode obtained by making various modifications conceivable by those skilled in the art to each embodiment; and a mode realized by combining together the components and the functions in each embodiment in any manner within a range not departing from the spirits of the present invention.

### REFERENCE MARKS IN THE DRAWINGS

10 air cleaning system
32 detection unit
34 airflow control unit
35 cleaning control unit
60 space

1. A space cleaning system, comprising:
a detection unit configured to detect generation and a generation position of an infectious substance in a space;
an airflow control unit configured to generate an airflow towards the generation position detected in response to detection of the generation of the infectious substance; and
a cleaning control unit configured to clean the space at least after the generation of the infectious substance is detected.

2. The space cleaning system according to claim 1, wherein
after the airflow is generated, the airflow control unit is configured to stop generation of the airflow after passage of a predetermined period.

3. The space cleaning system according to claim 1, wherein
the airflow control unit is configured to change a direction of the airflow after generating the airflow towards the generation position.

4. The space cleaning system according to claim 1, wherein
the cleaning control unit is configured to continuously clean the space from time before the generation of the infectious substance is detected to time after the generation of the infectious substance is detected.

5. The space cleaning system according to claim 4, wherein
the cleaning control unit is configured to increase a cleaning level to clean the space in response to the detection of the generation of the infectious substance.

6. The space cleaning system according to claim 1, wherein
the cleaning control unit is configured to start cleaning of the space after the generation of the infectious substance is detected.

7. The space cleaning system according to claim 1, wherein
the cleaning control unit is configured to control a device, which sterilizes air by use of hypochlorous acid, to clean the space.

8. The space cleaning system according to claim 1, wherein
the cleaning control unit is configured to control a device, which ventilates the space, to clean the space.

9. A space cleaning method, comprising:
detecting generation and a generation position of an infectious substance in a space;
generating an airflow towards the generation position detected in response to detection of the generation of the infectious substance; and
cleaning the space at least after the generation of the infectious substance is detected.
